(19) United States
(12) Patent Application Publication
(10) Pub. No.: US 2025/0040541 A1
(43) Pub. Date: Feb. 6, 2025

(54) AN ANTIMICROBIAL LIQUID COMPOSITION, PROCESS FOR PREPARING THE SAME AND METHOD OF USE THEREOF

(71) Applicant: ISP Investments LLC, Wilmington, DE (US)

(72) Inventors: Hani M. FARES, Somerset, NJ (US); Anthony ADAMO, Flagtown, NJ (US); Yevgeniy TUROVSKIY, Somerville, NJ (US)

(73) Assignee: ISP Investments LLC, Wilmington, DE (US)

(21) Appl. No.: 18/722,364

(22) PCT Filed: Dec. 19, 2022
(86) PCT No.: PCT/US2022/053331

§ 371 Date: Jun. 21, 2024

Related U.S. Application Data
(60) Provisional application No. 63/292,117, filed on Dec. 21, 2021.

(51) Int. Cl. A01N 35/02; A01P 1/00; A61K 8/34; A61K 8/35; A61Q 17/00

(52) U.S. Cl. CPC A01N 35/02; A01P 1/00; A61K 8/345; A61K 8/347; A61K 8/35; A61Q 17/005

(57) ABSTRACT

The present application provides an antibacterial liquid composition comprising: (a) from about 0.01 to about 99.8 wt. % of raspberry ketone having a structure of (I); (b) from about 0.1 to about 95% wt. % of one or more diols having a carbon chain length of from 3 to 12 atoms; and (c) from about 0.1 to about 95% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof. Also disclosed is a process for preparing the compositions and a method of use thereof.

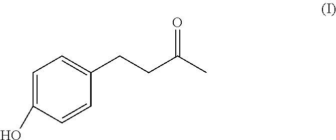

### FIELD OF THE INVENTION

The present application relates to an antibacterial liquid composition comprising: (a) raspberry ketone; (b) one or more diols having a carbon chain length of from 3 to 12 atoms; and (c) one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

### BACKGROUND OF THE INVENTION

Commercial use products are generally designed to have a substantial shelf life. The products need to be manufactured at one site, transported possibly over a considerable distance to a depot or other storage facility prior to further transport to a point of sale. The product may then spend considerable time on a retailer's shelf prior to purchase and further storage by the user whether for individual use or use in, for example, a workplace, institution or the like. Storage typically takes place under uncontrolled conditions including considerable variation in temperature. In order to keep bacterial and fungal growth in such products at an acceptable level, it is conventional practice for the products to contain a preservative. Many preservatives are available, the appropriate preservative needs to be selected based on its efficacy and its acceptability in the product.

Classic preservatives such as formaldehyde donors, parabens, and isothiazolinones are generally very effective at controlling microorganisms in various personal care products. However, in recent years, many consumers developed unfavorable perception of these chemistries. Current market trends strongly favor safe, non-irritating, natural/nature-identical, and biodegradable actives. As a result of these changes, there is a strong need for development of novel antimicrobial technologies for personal and home care applications. Chemistries that can boost antimicrobial performance of known actives are highly desired.

KR Patent 1881306B1 discloses cosmetics, personal care products, and home care and fabric care compositions comprising raspberry ketone, an alkyl-arginine derivative and 1,2-decanediol.

US Publication 20080317681 discloses a composition used as a chewing gum or confectionary for removing stains and microbes from teeth of warm-blooded animals having a stain removing complex containing stain removing agent and a cyclodextrin compound, and optionally a gum base.

US Publication 20170096381 discloses the use of vanillin derivatives in cosmetic, dermatological or pharmaceutical compositions.

PCT application No. 2010084661A1 discloses an isothiocyanate containing composition for treating insulin-like growth factor-1 associated diseases, e.g., hair loss and dementia, comprising isothiocyanates and one or more of isoflavone, raspberry ketone, capsiate and gluconic acid.

KR publication 2006034941A discloses an acaricidal composition is to control mites by selective insect killing and easy biodegradation of the compounds comprising at least one compound having acaricidal activity selected from 2-methoxyphenylacetone, 4-methoxyphenylacetone and 4-(4-methoxyphenyl)-2-butan-2-one.

In view of the foregoing, still there is a need for improved liquid antimicrobial compositions for aqueous or non-aqueous based end-user compositions for reducing, inhibiting or preventing microbial growth, comprising suitable and effective amounts of antimicrobial compositions in the desired end-user products.

Further, there is a huge demand for liquid antimicrobial compositions in view of ease of logistics/supply chain. There is a lack of literature on enhanced solubility of raspberry ketone. Raspberry Ketone is a solid, and generally, personal care industry has a strong preference for preservatives and preservative boosters in the liquid form, because liquids are easier to handle during the manufacturing process. Present invention is a solution of Raspberry Ketone—a liquid. Moreover, this liquid blend has a broad spectrum of antimicrobial activity and is effective as a preservative booster in a wide range of personal care applications.

Accordingly, it is an objective of the present invention to provide an antibacterial liquid composition comprising: (a) from about 0.01 to about 99.8 wt. % of raspberry ketone; (b) from about 0.1 to about 95% wt. % of one or more diols having a carbon chain length of from 3 to 12 atoms; and (c) from about 0.1 to about 95% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof. Also disclosed is a process for preparing the compositions and method of use thereof.

### SUMMARY OF THE INVENTION

The primary aspect of the present application is to provide an antibacterial liquid composition comprising: (a) from about 0.01 to about 99.8 wt. % of raspberry ketone having a structure of (I):

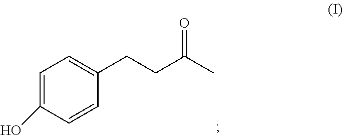

(b) from about 0.1 to about 95% wt. % of one or more diols having a carbon chain length of from 3 to 12 atoms; and (c) from about 0.1 to about 95% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

Another non-limiting aspect of the present application provides an antibacterial liquid composition can be a non-aqueous composition.

Another aspect of the present application provides a process for preparing antibacterial liquid composition, wherein the process comprising mixing of: (a) from about 0.01 to about 99.9 wt. % of raspberry ketone having a structure of (I):

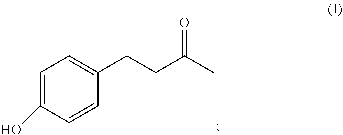

(b) from about 0.1 to about 95% wt. % of one or more diols having a carbon chain length of from 3 to 12 atoms; and (c) from about 0.1 to about 95% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

In another aspect, a method of killing or inhibiting the growth of bacteria and/or fungi in an aqueous or non-aqueous based end-user product selected from the group consisting of personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products that are susceptible to growth of microorganisms comprises incorporating the antimicrobial composition of the present application in an amount ranging from about 0.01 wt. % to 5.0 wt. % into the above desired products.

In yet another aspect, the present application provides an antibacterial liquid composition comprising: (a) from about 10 to about 40 wt. % of raspberry ketone having a structure of (I):

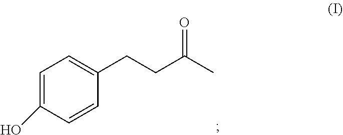

(b) from about 20 to about 60 wt. % of one or more diols having a carbon chain length of from 3 to 12 atoms; and (c) from about 20 to about 60 wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

Another aspect of the present application provides an antibacterial liquid composition comprising: (a) from about 10 to about 40 wt. % of raspberry ketone having a structure of (I):

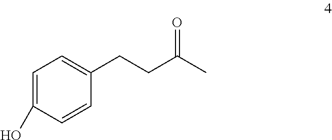

(b) from about 20 to about 60 wt. % of one or more diols selected from the group consisting of 1,2-hexanediol, 1,2-propanediol, 1,3-propanediol and combinations thereof; and (c) from about 20 to about 60 wt. % of one or more compounds selected from the group consisting of selected from the group consisting of phenylpropanol, phenethyl alcohol, benzyl alcohol and combinations thereof.

One more aspect of the present application provides an antibacterial liquid composition comprising: (a) about 25% of raspberry ketone having a structure of (I):

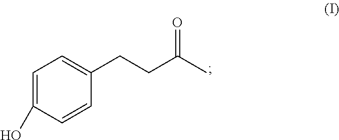

(b) about 37.5 wt. % of 1,3-propanediol; and (c) about 37.5 wt. % of phenylpropanol.

Another aspect of the present application provides an antibacterial liquid composition comprising: (a) about 25 wt. % of raspberry ketone having a structure of (I):

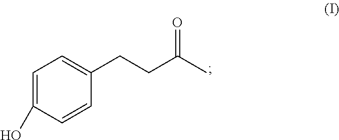

(b) about 37.5 wt. % of 1,3-propanediol; and (c) about 37.5 wt. % of benzylalcohol.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

As used herein, the term “antimicrobial” refers to substances capable of killing or inhibiting the growth of microorganisms, including but not limited, to bacteria and fungi.

As used herein, the term “preservative” refers to one or more compounds that are added to the composition to protect it against decay or decomposition or to help improve its shelf life.

As used herein, “Raspberry ketone”, also known as 4-(4-hydroxyphenyl) butan-2-one (HPB), having a CAS No: 5471-51-2, has been used as an aroma chemical in perfume industries, food industries and in compositions for weight loss with improved taste. Raspberry ketone was described for the first time as a characteristic component of Raspberry flavor (H. Schinz et. al. Helv. Chim. Acta. 1957, 40, 1839).

In a non-limiting embodiment, the present application discloses an antibacterial liquid composition comprising: (a) about 0.01 to about 99.8 wt. % of raspberry ketone having a structure of (I):

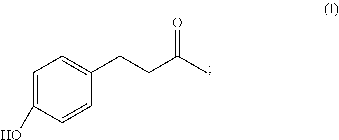

(b) about 0.1 to about 95% wt. % of one or more diols having a carbon chain length of from 3 to 12 atoms; and (c) about 0.1 to about 95% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

The suitable amounts of compounds of structure of (I) can be varied from about 0.01 wt. % to about 0.1 wt. %; or from 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. %; or from about 90 wt. % to about 95 wt. %; or from about 95 wt. % to about 99.8 wt. % based on the total weight of the antimicrobial composition.

According to the present application, non-limiting examples of suitable antimicrobial diols useful herein include, but are not limited to, propanediol, butanediol, pentanediol, hexanediol, octanediol, nonanediol, decanediol and dodecanediol.

In another non-limiting embodiments, it is contemplated to use diols having a carbon chain length of from 3 to 12 atoms, including but not limited to, propane-1,2-diol, propane-1,3-diol, butane-1,2-diol, butane-1,3-diol, butane-1,4-diol, 2-methylpropane-1,2-diol, 2-methylpropane-1,3-diol, pentane-1,2-diol, pentane-1,3-diol, pentane-1,4-diol, pentane-1,5-diol, pentane-2,3-diol, pentane-2,4-diol, 2-methyl-pentane-2,4-diol, hexane-1,2-diol, hexane-1,3-diol, hexane-1,4-diol, hexane-1,5-diol, hexane-1,6-diol, hexane-2,3-diol, hexane-2,4-diol, hexane-2,5-diol, hexane-3,4-diol, heptane-1,2-diol, heptane-1,3-diol, heptane-1,4-diol, heptane-1,5-diol, heptane-1,6-diol, heptane-1,7-diol, heptane-2,3-diol, heptane-2,4-diol, heptane-2,5-diol, heptane-2,6-diol, heptane-3,4-diol, heptane-3,5-diol, octane-1,2-diol, octane-1,3-diol, octane-1,4-diol, octane-1,5-diol, octane-1,6-diol, octane-1,7-diol, octane-1,8-diol, octane-2,3-diol, octane-2,4-diol, octane-2,5-diol, octane-2,6-diol, octane-2,7-diol, octane-3,4-diol, octane-3,5-diol, octane-3,6-diol and octane-4,5-diol, nonane-1,9 diol, decane-1,2-diol, decane-1,10-diol, hexadecan-1-ol, and dodecane-1,12-diol.

In another non-limiting embodiment, the diols have a length of from 3 to 12 carbon atoms, including but not limited to, pentane-1,2-diol, hexane-1,2-diol, and octane-1,2-diol.

In one of the alternative embodiment, the composition comprising a conventional preservative compounds selected from the group including, but not limited to, benzoic acid and its sodium salt such as benzoic acid, sodium benzoate; salts of benzoic acid such as ammonium benzoate, butyl benzoate, calcium benzoate, ethyl benzoate, isobutyl benzoate, isopropyl benzoate, magnesium benzoate, MEA-benzoate, methyl benzoate, phenyl benzoate, potassium benzoate, propyl benzoate; propanoic acid and its salts such as propionic acid, ammonium propionate, calcium propionate, magnesium propionate, potassium propionate, sodium propionate; salicylic acid and its salts such as salicylic acid, calcium salicylate, magnesium salicylate, MEA-salicylate, sodium salicylate, potassium salicylate, TEA-salicylate; hexa-2,4-dienoic acid and its salts such as sorbic acid, calcium sorbate, sodium sorbate, potassium sorbate; biphenyl-2-ol as o-phenylphenol; inorganic sulphites and hydrogen sulphites such as sodium sulfite, ammonium bisulfite, ammonium sulfite, potassium sulfite, potassium hydrogen sulfite, sodium bisulfite, sodium meta bisulfite, potassium meta bisulfite; chlorobutanol; 4-hydroxybenzoic acid and its salts and esters other than the esters of isopropyl, isobutyl, phenyl, benzyl and pentyl such as 4-hydroxybenzoic acid, methylparaben, potassium ethylparaben, potassium paraben, sodium methylparaben, sodium ethylparaben, ethylparaben, sodium paraben, potassium methylparaben, calcium paraben; butyl 4-hydroxybenzoate and its salts and propyl 4-hydroxybenzoate and its salts such as butylparaben, propylparaben, sodium propoylparaben, sodium butylparaben, potassium butylparaben, potassium propylparaben; 3-acetyl-6-methylpyran-2,4 (3H)-dione and its salts such as dehydroacetic acid, sodium dehydroacetate; formic acid, sodium formate; 3,3′-dibromo-4,4′-hexamethylene dioxydibenzamidine and its salts (including isethionate) such as dibromohexamidine isethionate; thimerosal; phenylmercuric salts such as phenyl mercuric acetate, phenyl mercuric benzoate; undec-10-enoic acid and its salts such as undecylenic acid, potassium undecylenate, sodium undecylenate, calcium undecylenate, MEA-undecylenate, TEA-undecylenate; 5-pyrimidinamine 1,3-bis(2-ethylhexyl) hexahydro-5-methyl-such as hexetidine; 5-bromo-5-nitro-1,3-dioxane; bronopol such as 2-bromo-2-nitropropane-1,3-diol; 2,4-dichlorobenzyl alcohol; 1-(4-chlorophenyl)-3-(3,4-dichlorophenyl) urea such as triclocarban; chlorocresol such as p-chloro-m-cresol, 5-chloro-2-(2,4-dichlorophenoxy) phenol such as triclosan; chloroxylenol; N,N″-methylenebis[N′-[3-(hydroxymethyl)-2,5-dioxoimidazolidin-4-yl]urea] such as imidazolidinyl urea; polyhexamethylene biguanide hydrochloride such as polyaminopropyl biguanide; 2-phenoxyethanol; methenamine; 1-(4-chlorophenoxy)-1-(imidazol-1-yl)-3,3-dimethylbutan-2-one such as climbazole; 1,3-bis(hydroxymethyl)-5,5-dim ethylimidazolidine-2,4-dione such as DMDM hydantoin; benzyl alcohol; 1-hydroxy-4-methyl-6-(2,4,4-tri methylpentyl)-2 pyridon and its monoethanolamine salt such as 1-hydroxy-4-methyl-6-(2,4,4-trimethylpentyl)-2pyridon, piroctone olamine; 2,2′-methylenebis(6-bromo-4-chlorophenol) such as bromochlorophene; 4-isopropyl-m-cresol as o-cymen-5-ol; mixture of 5-chloro-2-methyl-isothiazol-3 (2H)-one and 2-methylisothiazol-3 (2H)-one with magnesium chloride and magnesium nitrate as methylchloroisothiazolinone and methylisothiazolinone; 2-chloroacetamide; N,N′-bis(4-chlorophenyl)-3,12-diimino-2,4,11,13-tetraazatetradeca nediamidine and its digluconate, diacetate and dihydrochloride such as chlorhexidine, chlorhexidine diacetate, chlorhexidine digluconate, chlorhexidine dihydrochloride; 1-phenoxypropan-2-ol such as phenoxyisopropanol; alkyl (C12-C22) trimethyl ammonium bromide and chloride such as behentrimonium chloride, cetrimonium bromide, cetrimonium chloride, laurtrimonium bromide, laurtrimonium chloride, steartrimonium bromide, steartrimonium chloride; 4,4-dimethyl-1,3-oxazolidine; N-(hydroxymethyl)-N-(dihydroxymethyl-1,3-dioxo-2,5-imidazolidinyl-4)-N′-(hydroxymethyl) urea such as diazolidinyl urea; benzenecarboximidamide 4,4′-(1,6-hexanediylbis(oxy))bis- and its salts (including isothionate and p-hydroxybenzoate) such as hexamidine, hexamidine diisethionate, hexamidine diparaben, hexamidine paraben; glutaraldehyde (pentane-1,5-dial) such as glutaral; 5-ethyl-3,7-dioxa-1-azabicyclo[3.3.0]octane such as 7-ethylbicyclooxazolidine; 3-(p-chlorophenoxy)-propane-1,2-diol such as chlorphenesin; sodium hydroxymethylamino acetate such as sodium hydroxymethylglycinate; silver chloride deposited on titanium dioxide; benzethonium chloride such as benzenemethanaminium, N,N-dimethyl-N-[2-[2-[4-(1,1,3,3,-tetramethylbutyl) phenoxy]ethoxy]ethyl]-chloride; benzalkonium chloride, benzalkonium bromide, benzalkonium saccharinate; methanol, (phenylmethoxy) such as benzylhemiformal; 3-iodo-2-propynylbutylcarbamate; 2-methyl-2H-isothiazol-3-one; ethyl lauroyl arginate HCl; citric acid (and) silver citrate such as 1,2,3-propanetricarboxylic acid, 2-hydroxy-, monohydrate and 1,2,3-propanetricarboxylic acid, 2-hydroxy-silver (1+) salt, monohydrate; and 4-(3-ethoxy-4-hydroxyphenyl) bu-tan-2-one.

In one of the alternative embodiment, the composition comprising one or more antimicrobial agents useful herein include one or more antimicrobial compounds selected from the group consisting of propane-1,3-diol, pentane-1,2-diol, hexane-1,2-diol, octane-1,2-diol, hexadecan-1-ol, citric acid, stearic acid, benzoic acid, anisic acid, cinnamic acid, phytic acid, caprylhydroxamic acid, hinokitiol, ethylhexylglycerin, hexyl glycerin, glyceryl caprylate/caprate, veratraldehyde, maltol, ethyl maltol, phenyl propanol, tetradecyl trimethyl ammonium bromide (TTAB), 3-iodo-2-propynylbutyl-carbamate (IPBC), sodium fluoride and combinations thereof.

In one of the alternative embodiment, the suitable range of one or more antimicrobial compounds not being compounds of structure of (I) of the present application can be varied from about 0.01 wt. % to about 0.1 wt. %; or from 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. %; from about 90 wt. % to about 95 wt. % or from about 95 wt. % to about 99.8 wt. % based on the total weight of the antimicrobial composition.

The antimicrobial composition of the present application is useful for inhibiting or killing Staphylococcus aureus, Staphylococcus epidermidis, Streptococcus pneumoniae, Streptococcus pyogenes, Enterococcus faecalis, Haemophilus influenzae, Moraxella species, salmonella species, Campylobacter species, Pseudomonas aeruginosa, Clostridium botulinum, Clostridium perfringens, Corynebacteria species, Diplococci species, Mycobacteria species, Streptomyces species, Escherichia coli, Salmonella typhimurium, Salmonella enteritidis, Vibrio parahaemolyticus, Bacillus anthracis, Bacillus azotoformans, Bacillus cereus, Bacillus coagulans, Bacillus israelensis, Bacillus larvae, Bacillus mycoides, Bacillus polymyxa, Bacillus pumilis, Bacillus stearothormophillus, Bacillus subtilis, Bacillus thuringiensis, Bacillus validus, Bacillus weihenstephanensis, Bacillus pseudomycoides, Burkholderia cepacia, Burkholderia multivorans, Burkholderia cenocepacia, Burkholderia vietnamiensis, Burkholderia stabilis, Burkholderia ambifaria, Burkholderia dolosa, Burkholderia anthina, Burkholderia pyrrocinia, Candida tropicalis, Candida albicans, Hansemula anomala, Saccharomyces cerevisiae, Torulaspora delbreuckii, Zygosaccharomyces bailii, Zygosaccharomyces rouxii, Aspergillus niger, Aspergillus flavus, Aspergillus brasiliensis, Penicillium islandicum, Penicillium citrinum, Penicillium chrysogenum, Fusarium oxysporum, Fusarium graminearum, Fusarium solani, Alternaria alternata, and or Mucor racemosus.

According to a non-limiting embodiment of the present application, the antimicrobial composition is used for killing or inhibiting the growth of Staphylococcus aureus, Escherichia coli, Burkholderia cepacia, Candida albicans, Pseudomonas aeruginosa, and Aspergillus brasiliensis.

A different embodiment of the present application contemplates that the antimicrobial composition of the present application can be formulated as an emulsion, microemulsion, nanoemulsion, solution, dispersion, suspension, complex coacervate, or concentrate. The antimicrobial compositions can also include various optional additives. Examples of specific additives include, but are not limited to, colorants, pigments, plasticizers, surfactants, wetting agents, fillers, coloring agents, dispersing agents, thickening agents, rheology modifying agents, thixotropic agents, anti-freezing agents, co-solvents, pH modifying agents, ultraviolet light stabilizers, antioxidants, algaecides, antimicrobial agents, fragrances, buffers, hydrotropes, anti-soil agents, enzymes, suspending agents, emulsifying agent, anti-foaming agents, organic solvents, VOC-free solvents, solubilizers, and/or water-miscible solvents.

According to another non-limiting embodiment of the present application, the composition is a non-aqueous composition.

According to another non-limiting embodiment of the present application, the composition is formulated as an emulsion, microemulsion, nanoemulsion, solution, dispersion, suspension, complex coacervate or concentrate.

According to another non-limiting embodiment of the present application, the antimicrobial composition can provide a synergistic effect in various aqueous and non-aqueous based end-user applications, and wherein the synergistic index (SI) value is greater than 0.1 to less than 1.

Another non-limiting embodiment of the present application discloses that the antimicrobial composition can advantageously be used in personal care compositions, and wherein, the compositions can be an aqueous or non-aqueous based end-user composition. Aqueous and non-aqueous based end-user applications include, but are not limited to, personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products.

According to another non-limiting embodiment of the present application, the personal care compositions include, but are not limited to, sun care compositions, after-sun compositions, hair care compositions, conditioning compositions, skin care compositions, oral care compositions, face care compositions, lip care compositions, body care compositions, nail care compositions, anti-aging compositions, deodorant compositions, color cosmetic compositions, color-protection compositions, self-tanning compositions, and foot care compositions.

In non-limiting embodiments, the present application discloses that suitable ranges of incorporating the above-described antimicrobial composition for killing or inhibiting the growth of bacteria and fungi in aqueous or non-aqueous based end-user products can be varied from about 0.01 wt. % to about 0.1 wt. %, or from about 0.1 wt. % to about 1 wt. %, or from about 1 wt. % to about 2.5 wt. %, or from about 2.5 wt. % to about 5 wt. % based on the total weight of the aqueous or non-aqueous personal care composition.

According to another non-limiting embodiment of the present application, the present application discloses a process for preparing antibacterial liquid composition, wherein the process comprising mixing of: (a) from about 0.01 to about 99.9 wt. % of raspberry ketone having a structure of (I):

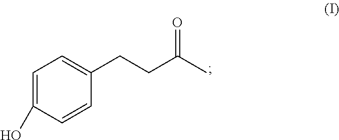

(b) from about 0.1 to about 95% wt. % of one or more diols having a carbon chain length of from 3 to 12 atoms; and (c) from about 0.1 to about 95% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

Yet another embodiment of the present application discloses a method of killing or inhibiting the growth of bacteria and fungi, in an aqueous or non-aqueous based end-user products selected from the group consisting of personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products that are susceptible to growth of microorganisms comprising incorporating from about 0.01 wt. % to 5.0 wt. % of the antimicrobial composition into said products.

In non-limiting embodiments, the present application discloses an antibacterial liquid composition comprising: (a) from about 10 to about 40 wt. % of raspberry ketone having a structure of (I):

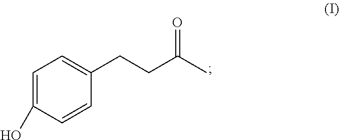

(b) from about 20 to about 60% wt. % of one or more diols having a carbon chain length of from 3 to 12 atoms; and (c) from about 20 to about 60% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

Yet another embodiment of the present application discloses an antibacterial liquid composition comprising: (a) about 25% of raspberry ketone having a structure of (I):

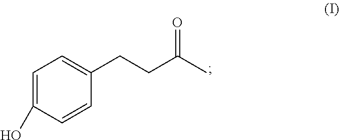

(b) about 37.5 wt. % of 1,3-propanediol; and (c) about 37.5 wt. % of phenylpropanol.

In another non-limiting embodiments, the present application discloses an antibacterial liquid composition comprising: (a) about 25 wt % of raspberry ketone having a structure of (I):

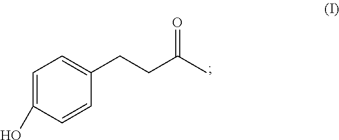

(b) about 37.5 wt. % of 1,3-propanediol; and (c) about 37.5 wt. % of benzylalcohol.

Further, certain aspects of the present application are illustrated in detail by way of the following examples. The examples are given herein for illustration of the application and are not intended to be limiting thereof.

### EXAMPLES

### Example 1 Demonstrates Solubility of Raspberry Ketone in the Two-Component Solvent System Resulting in the Antimicrobial Blend with the Broad Spectrum of Activity

Method: Solubility of raspberry ketone in various solvents was determined by adding 15%, 20%, 25%, 30% and 35% wt. % of raspberry ketone to a solvent and stirring it at 60° C. If the solution became clear, it was then split into two containers to be stored overnight at 4° C. and 25° C. to evaluate its stability. The next day, both containers were visually examined for any signs of crystallization. The highest concentration of raspberry ketone that formed a clear solution which remained stable after storage at both 4° C. and 25° C. was deemed as the solubility of raspberry ketone in a particular solvent. For the two-component solvent systems, solvents were mixed in 1:1 ratio (W/W).

TABLE 1
the solubility of raspberry ketone in various solvents

| | Raspberry ketone |
|---|---|
| Solvent | solubility (wt. %) |
| Propylene Glycol | 15% |
| Propanediol | 15% |
| Butylene Glycol | 15% |
| Pentylene Glycol | <15% |
| Hexylene Glycol | 20% |
| Caprylyl Glycol | <15% |
| Phenethyl Benzoate | <15% |
| Phenethyl Alcohol | 25% |
| Phenylpropanol | 25% |
| Benzyl Alcohol | 25% |
| Glyceryl Caprylate | <15% |

TABLE 2
the solubility of 25% raspberry ketone in the two-component solvent
systems containing 1:1 ratio of each component (wt. %)

| | Propylene | | Butylene | Pentylene | Hexylene |
|---|---|---|---|---|---|
| | Glycol | Propanediol | Glycol | Glycol | Glycol |
| Phenethyl | Yes | Yes | Yes | No | Yes |
| Alcohol | | | | | |
| Phenyl | Yes | Yes | Yes | No | Yes |
| propanol | | | | | |
| Benzyl | Yes | Yes | Yes | Yes | Yes |
| Alcohol | | | | | |
| Glyceryl | No | No | No | No | No |
| Caprylate | | | | | |

Raspberry ketone has a very limited solubility in aliphatic solvents acceptable in personal care (≤20% wt. %). In contrast, raspberry ketone raspberry ketone is soluble at 25 wt. % in aromatic solvents (Table 1). Unexpectedly, raspberry ketone forms stable 25% wt. % solutions in 1:1 mixture of aromatic phenylpropanol and some of the aliphatic diols (Table 2). This invention is especially significant, because inclusion of both aliphatic and aromatic solvents with antimicrobial properties is likely to result in broad antimicrobial activity of the overall blend.

### Example 2: Composition of the Blend #1 Used in Example 2

| Component | wt. % |
|---|---|
| Raspberry ketone | 25 |
| Phenylpropanol | 37.5 |
| 1,3-Propanediol | 37.5 |

Data demonstrating synergy between raspberry ketone and other components of the blend was generated following a 28-day double inoculation challenge test. The test was conducted in sunscreen emulsion base, pH 7 (Table 3).

TABLE 3
composition of the sunscreen emulsion base used in Example 2

| Phase | INCI | wt. % |
|---|---|---|
| A | Water | 56.6 |
| | Propylene glycol | 2 |
| | Hydroxyethylcellulose | 0.2 |
| | Cellulose | 0.4 |
| | antimicrobial active | 2 |
| B | Avobenzone | 3 |
| | Ethylhexyl Salicylate | 5 |
| | Octocrylene | 5 |
| | Benzophenone-3 | 6 |
| | Homosalate | 10 |
| | Cetyl alcohol | 1.5 |
| | Cetearyl Alcohol (and) Dicetyl | 4 |
| | Phosphate (and) Ceteth-10 | |
| | Phosphate | |
| | Myristyl Myristate (and) Myristyl | 2 |
| | Laurate | |
| | Diisopropyl Adipate | 2 |
| C | pH Adjust, 20% Sodium | 0.3 |
| | Hydroxide | |
| | Total | 100 |

Method: The emulsion was formulated with 2% of the blend #1 or with the appropriate controls, plus water to add up to the 2% of the concentration in the emulsion.

Briefly, each tested sample was split into three containers to be challenged, respectively, with either of the three classes of common contaminants: Gram-positive bacteria, Gram-negative bacteria, and fungi. Accordingly, three types of inocula were prepared as outlined in the Table 4. The Gram-positive bacterial inoculum consisted of the single microorganism, while the Gram-negative bacterial inoculum and fungal inoculum, each, consisted of more than one species pooled together (Table 4).

TABLE 4
the microbial composition of each
inocula used in the challenge test

| Inoculum class | Microbial species |
|---|---|
| Gram-positive bacteria | Staphylococcus aureus 6538 |
| Gram-negative bacteria | Escherichia coli 8739, |
| | Pseudomonas aeruginosa 9027, |
| | Burkholderia cepacia 25416 |
| Fungi | Candida albicans 10231 and |
| | Aspergillus brasiliensis 16404 |

The test samples were spiked with the microbial inoculum on days 0 and 21, with the final concentration of bacteria being 10⁶ cfu/g and the final concentration of fungi being 10⁵ cfu/g. Inoculated samples were incubated at ambient temperature. On days 2, 7, 21 and 28 the samples were plated to recover viable microorganisms. Letheen Agar was used for the recovery of bacteria and on Potato Dextrose Agar was used for the recovery of fungi.

TABLE 5
plate counts (cfu/g) recovered from each formula after being
challenged with microorganisms on day 0 and day 21:

| Compositions | pH | Organism(s) | 2 days | 7 days | 21 days* | 28 days |
|---|---|---|---|---|---|---|
| Control, no | | Gram+ bac | >10⁴ | <10 | <10 | 9.40 × 10³ |
| active | | Gram− bac | >10⁴ | >10⁴ | >10⁴ | >10⁴ |
| 2% water | | Fungi | 3.60 × 10⁴ | 3.70 × 10⁴ | >10⁴ | >10⁴ |
| 2.0% Blend of | 7 | Gram+ bac | <10 | <10 | <10 | <10 |
| Example #1 | | Gram− bac | <10 | <10 | <10 | <10 |
| | | fungi | 10⁴ | <10 | <10 | <10 |
| 0.75% | 7 | Gram+ bac | >10⁴ | <10 | <10 | 1.30 × 10³ |
| Phenylpropanol, | | Gram− bac | >10⁴ | >10⁴ | 6.00 × 10³ | >10⁴ |
| 1.25% water | | Fungi | 3.70 × 10⁴ | 1.80 × 10⁴ | 4.00 × 10³ | >10⁴ |
| 0.5% Raspberry | 6.99 | Gram+ bac | >10⁴ | <10 | <10 | 2.1 × 10² |
| ketone, | | Gram− bac | >10⁴ | >10⁴ | 1.3 × 10³ | >10⁴ |
| 1.5% water | | fungi | 2.4 × 10⁴ | 2.40 × 10⁴ | 4.4 × 10³ | >10⁴ |
| 0.75% 1,3- | 6.99 | Gram+ bac | >10⁴ | <10 | <10 | 2.50 × × 10² |
| Propanediol, | | Gram− bac | >10⁴ | <10 | <10 | 2.00 × 10² |
| 1.25% water | | Fungi | 3.40 × 10⁴ | 1.6 × 10⁴ | 9.5 × 10⁴ | >10⁴ |
| 0.75% 1,3- | 6.93 | Gram+ bac | 2.5 × 10³ | <10 | <10 | 1.00 × 10² |
| Propanediol, | | Gram− bac | 4 × 10² | <10 | <10 | 3 × 10¹ |
| 0.75% | | Fungi | 1 × 10⁴ | <10 | <10 | <10 |
| Phenylpropanol, | | | | | | |
| 0.5% water | | | | | | |

*Samples were re-inoculated on day 21, after being plated
Gram+ bac: Gram-positive bacteria
Gram− bac: Gram-negative bacteria

Results of the preservative challenge test are outlined in Table 5. The blend #1 clearly outperforms the individual components of the blend and the mixture of phenylpropanol and 1,3-propanediol in corresponding concentrations. In the formulation preserved with blend #1, bacteria were undetectable (<10 cfu/g) two days following the first inoculation. Fungi were undetectable (<10 cfu/g) seven days following the first inoculation. Also, no microorganisms were detected on day 28 (7 days following the second inoculation).

TABLE 6
plate counts (cfu/g) recovered from each formula after being
challenged with microorganisms on day 0 and day 21:

| | pH | Organism(s) | 2 days | 7 days | 21 days* | 28 days |
|---|---|---|---|---|---|---|
| 1.5% | 7 | Gram+ bac | 7.2 × 10³ | <10 | <10 | <10 |
| Blend#1 | | Gram− bac | 4 × 10¹ | <10 | <10 | <10 |
| | | fungi | 1 × 10⁴ | <10 | <10 | <10 |
| 1.0% | 6.9 | Gram+ bac | >10⁴ | <10 | <10 | <10 |
| Blend#1 | | Gram− bac | 2.4 × 10⁴ | <10 | <10 | <10 |
| | | Fungi | 1 × 10⁴ | 1 × 10² | <10 | <10 |

*Samples were re-inoculated on day 21, after being plated

Table 6 demonstrates that the blend #1 also adequately preserves the formulation at lower concentrations of 1.0% and 1.5%.

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

What is claimed is:

1. An antibacterial liquid composition comprising:
(a) from about 0.01 to about 99.8 wt. % of raspberry ketone having a structure of (I): 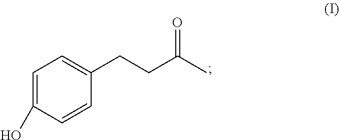
(b) from about 0.1 to about 95% wt. % of one or more diols having a carbon chain length of from 3 to 12 atoms; and
(c) from about 0.1 to about 95% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

2. The antibacterial liquid composition according to claim 1, wherein the diols are selected from the group consisting of propane-1,2-diol, propane-1,3-diol, butane-1,2-diol, butane-1,3-diol, butane-1,4-diol, 2-methylpropane-1,2-diol, 2-methylpropane-1,3-diol, pentane-1,2-diol, pentane-1,3-diol, pentane-1,4-diol, pentane-1,5-diol, pentane-2,3-diol, pentane-2,4-diol, 2-methyl-pentane-2,4-diol, hexane-1,2-diol, hexane-1,3-diol, hexane-1,4-diol, hexane-1,5-diol, hexane-1,6-diol, hexane-2,3-diol, hexane-2,4-diol, hexane-2,5-diol, hexane-3,4-diol, heptane-1,2-diol, heptane-1,3-diol, heptane-1,4-diol, heptane-1,5-diol, heptane-1,6-diol, heptane-1,7-diol, heptane-2,3-diol, heptane-2,4-diol, heptane-2,5-diol, heptane-2,6-diol, heptane-3,4-diol, heptane-3,5-diol, octane-1,2-diol, octane-1,3-diol, octane-1,4-diol, octane-1,5-diol, octane-1,6-diol, octane-1,7-diol, octane-1,8-diol, octane-2,3-diol, octane-2,4-diol, octane-2,5-diol, octane-2,6-diol, octane-2,7-diol, octane-3,4-diol, octane-3,5-diol, octane-3,6-diol and octane-4,5-diol, nonane-1,9 diol, decane-1,2-diol, decane-1,10-diol, hexadecan-1-ol, dodecane-1,12-diol, and combinations thereof.

3. The antibacterial liquid composition according to claim 1, wherein the composition is used for killing or inhibiting the growth of Staphylococcus aureus, Staphylococcus epidermidis, Streptococcus pneumoniae, Streptococcus pyogenes, Enterococcus faecalis, Haemophilus influenzae, Moraxella species, salmonella species, Campylobacter species, Pseudomonas aeruginosa, Clostridium botulinum, Clostridium perfringens, Corynebacteria species, Diplococci species, Mycobacteria species, Streptomyces species, Escherichia coli, Salmonella typhimurium, Salmonella enteritidis, Vibrio parahaemolyticus, Bacillus anthracis, Bacillus azotoformans, Bacillus cereus, Bacillus coagulans, Bacillus israelensis, Bacillus larvae, Bacillus mycoides, Bacillus polymyxa, Bacillus pumilis, Bacillus stearothormophillus, Bacillus subtilis, Bacillus thuringiensis, Bacillus validus, Bacillus weihenstephanensis, Bacillus pseudomycoides, Burkholderia cepacia, Burkholderia multivorans, Burkholderia cenocepacia, Burkholderia vietnamiensis, Burkholderia stabilis, Burkholderia ambifaria, Burkholderia dolosa, Burkholderia anthina, Burkholderia pyrrocinia, Candida tropicalis, Candida albicans, Hansenula anomala, Saccharomyces cerevisiae, Torulaspora delbreuckii, Zygosaccharomyces bailii, Zygosaccharomyces rouxii, Aspergillus niger, Aspergillus flavus, Aspergillus brasiliensis, Penicillium islandicum, Penicillium citrinum, Penicillium chrysogenum, Fusarium oxysporum, Fusarium graminearum, Fusarium solani, Alternaria alternata and or Mucor racemosus.

4. The antibacterial liquid composition according to claim 1, wherein the composition is used for killing or inhibiting the growth of Staphylococcus aureus, Escherichia coli, Burkholderia cepacia, Candida albicans, Pseudomonas aeruginosa and Aspergillus brasiliensis.

5. The antibacterial liquid composition according to claim 1, wherein the composition is a non-aqueous composition.

6. The antibacterial liquid composition according to claim 1, wherein the composition is formulated as an emulsion, microemulsion, nanoemulsion, solution, dispersion, suspension, complex coacervate or concentrate.

7. The antibacterial liquid composition according to claim 1, wherein the composition can provide synergistic effect having a synergistic index (SI) value greater than 0.1 to less than 1.

8. Use of antibacterial liquid composition according of claim 1, in an aqueous and non-aqueous based end-user applications selected from the group consisting of personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products.

9. The use of antibacterial liquid composition according to claim 8, wherein the personal care or cosmetic composition includes sun care compositions, after-sun compositions, hair care compositions, conditioning compositions, skin care compositions, oral care compositions, face care compositions, lip care compositions, body care compositions, nail care compositions, anti-aging compositions, deodorant compositions, color cosmetic compositions, color-protection compositions, self-tanning compositions and foot care compositions.

10. The use of antibacterial liquid composition according to claim 9, wherein the amount of said composition employed in an aqueous or non-aqueous based end-user composition is in the range of from about 0.01 wt. % to about 5.0 wt. % of the total composition.

11. A process for preparing antibacterial liquid composition according of claim 1, wherein the process comprising mixing of:
(a) from about 0.01 to about 99.9 wt. % of raspberry ketone having a structure of (I): 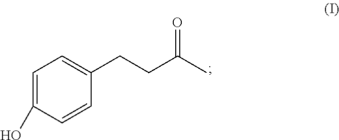
(b) from about 0.1 to about 95% wt. % of one or more diols having a carbon chain length of from 3 to 12 atoms; and
(c) from about 0.1 to about 95% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

12. A method of killing or inhibiting the growth of bacteria and fungi, in an aqueous or non-aqueous based end-user products selected from the group consisting of personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products that are susceptible to growth of microorganisms comprising incorporating about 0.01 wt. % to 5.0 wt. % of the antimicrobial composition of claim 1 into said products.

13. An antibacterial liquid composition comprising:
(a) from about 10 to about 40 wt. % of raspberry ketone having a structure of (I): 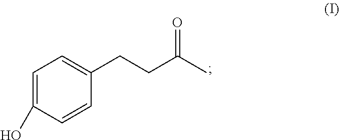
(b) from about 20 to about 60% wt. % of one or more diols having a carbon chain length of from 3 to 12 atoms; and
(c) from about 20 to about 60% wt. % of one or more compounds selected from the group consisting of phenoxyethanol, phenoxypropanol, phenylpropanol, phenethyl alcohol, veratryl alcohol, benzyl alcohol, 2-methyl-1-phenyl-2-propanol and combinations thereof.

14. An antibacterial liquid composition comprising:
(a) from about 10 to about 40 wt. % of raspberry ketone having a structure of (I): 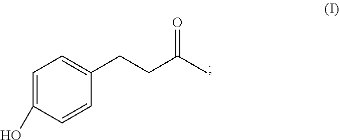
(b) from about 20 to about 60% wt. % of one or more diols selected from the group consisting of 1,2-hexanediol, 1,2-propanediol, 1,3-propanediol and combinations thereof; and
(c) from about 20 to about 60% wt. % of one or more compounds selected from the group consisting of phenylpropanol, phenethyl alcohol, benzyl alcohol and combinations thereof.

15. An antibacterial liquid composition comprising:
(a) about 25% of raspberry ketone having a structure of (I): 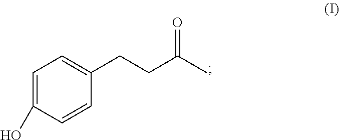
(b) about 37.5 wt. % of 1,3-propanediol; and
(c) about 37.5 wt. % of phenylpropanol.

16. An antibacterial liquid composition comprising:
(a) about 25% of raspberry ketone having a structure of (I): 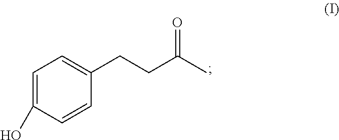
(b) about 37.5 wt. % of 1,3-propanediol; and
(c) about 37.5 wt. % of benzylalcohol.
